(19) United States
(12) Patent Application Publication
(10) Pub. No.: US 2022/0008566 A1
(43) Pub. Date: Jan. 13, 2022

(54) Pharmaceutical Composition Comprising a Radiolabeled GRPR Antagonist and a Surfactant

(71) Applicant: Advanced Accelerator Applications International SA, Geneva (CH)

(72) Inventors: Francesca Orlandi, Ivrea (Turin) (IT); Lorenza Fugazza, Ivrea (Turin) (IT); Donato Barbato, Ivrea (Turin) (IT); Mattia Tedesco, Alba (Cuneo) (IT); Lorenzo Sacchetti, Torino (Turin) (IT)

(73) Assignee: Advanced Accelerator Applications International SA, Geneva (CH)

(21) Appl. No.: 17/283,462

(22) PCT Filed: Oct. 11, 2019
(86) PCT No.: PCT/EP2019/077569

§ 371 Date: Apr. 7, 2021

(30) Foreign Application Priority Data
Oct. 12, 2018 (EP) 18200246.9

(51) Int. Cl. A61K 51/08; A61K 47/14; A61K 51/12; A61K 47/34

(52) U.S. Cl. CPC A61K 51/088; A61K 47/34; A61K 51/121; A61K 47/14

(57) ABSTRACT

The present disclosure relates to gastrin-releasing peptide receptor (GRPR) targeting radiopharmaceuticals and uses thereof. In particular, the present disclosure relates to a pharmaceutical composition comprising radiolabeled GRPR-antagonist and a surfactant. The present disclosure also relates to radiolabeled GRPR-antagonist for use in treating or preventing a cancer.

### TECHNICAL FIELD

The present disclosure relates to gastrin-releasing peptide receptor (GRPR) targeting radiopharmaceuticals and uses thereof. In particular, the present disclosure relates to a pharmaceutical composition comprising radiolabeled GRPR-antagonist and a surfactant. The present disclosure also relates to radiolabeled GRPR-antagonist for use in treating or preventing a cancer.

### BACKGROUND ART

The gastrin-releasing peptide receptor (GRPR), also known as bombesin receptor subtype 2, is a G-protein-coupled receptor expressed in various organs, including those of the gastrointestinal tract and the pancreas (Guo M, et al. Curr Opin Endocrinol Diabetes Obes. 2015; 22:3-8,2; Gonzalez N, et al. Curr Opin Enocrinol Diabetes Obes. 2008; 15:58-64). On binding of a suitable ligand, the GRPR is activated, eliciting multiple physiologic processes, such as regulation of exocrine and endocrine secretion (Guo M, et al. Curr Opin Endocrinol Diabetes Obes. 2015; 22:3-8,2; Gonzalez N, et al. Curr Opin Enocrinol Diabetes Obes. 2008; 15:58-64). In the past decades, GRPR expression has been reported in various cancer types, including prostate cancer and breast cancer (Gugger M and Reubi J C. Gastrin-releasing peptide receptors in non-neoplastic and neoplastic human breast. Am J Pathol. 1999; 155:2067-2076; Markwalder R and Reubi J C. Cancer Res. 1999; 59:1152-1159). Therefore, the GRPR became an interesting target for receptor-mediated tumor imaging and treatment, such as peptide receptor scintigraphy and peptide receptor radionuclide therapy (Gonzalez N, et al. Curr Opin Enocrinol Diabetes Obes. 2008; 15:58-64). After the successful use of radiolabeled somatostatin peptide analogs in neuroendocrine tumors for nuclear imaging and therapy (Brabander T, et al. Front Horm Res. 2015; 44:73-87; Kwekkeboom D J and Krenning E P. Hematol Oncol Clin North Am. 2016; 30:179-191), multiple radiolabeled GRPR radioligands have been synthesized and studied in preclinical as well as in clinical studies, mostly in prostate cancer patients. Examples of such peptide analogs include AMBA, the Demobesin series, and MP2653 (Yu Z, et al. Curr Pharm Des. 2013; 19:3329-3341; Lantry L E, et al. J Nucl Med. 2006; 47:1144-1152.; Schroeder R P et al. Eur J Nucl Med Mol Imaging. 2010; 37:1386-1396.; Nock B, et al. Eur J Nucl Med Mol Imaging. 2003; 30:247-258.; Mather S J, et al. Mol Imaging Biol. 2014; 16:888-895). Recent studies have shown a preference for GRPR antagonists compared with GRPR agonists (Mansi R, et al. Eur J Nucl Med Mol Imaging. 2011; 38:97-107; Cescato R, et al. J Nucl Med. 2008; 49:318-326). Compared with receptor agonists, antagonists often show higher binding and favorable pharmacokinetics (Ginj M, et al. Proc Natl Acad Sci USA. 2006; 103:16436-16441). Also, clinical studies with radiolabeled GRPR agonists reported unwanted side effects in patients caused by activation of the GRPR after binding of the peptide to the receptor (Bodei L, et al. [abstract]. Eur J Nucl Med Mol Imaging. 2007; 34:S221).

It was recently found that some GRPR-antagonists, like NeoBOMB1, can be radiolabeled with different radionuclides and could potentially be used for imaging and for treating GRPR-expressing cancers, for example but not limited to, prostate cancer and breast cancer. However, only biodistribution studies have been reported so far and no efficient treatment protocol or pharmaceutical compositions have been developed.

In this context, it would thus be desirable to provide a pharmaceutical composition comprising a GRPR-antagonist which could be administrated to patients. Moreover, it would also be desirable to provide an efficient treatment protocol for patients with cancer using a GRPR-antagonist.

### SUMMARY

In a first aspect, the present disclosure relates to pharmaceutical composition comprising
a radiolabeled GRPR-antagonist of the following formula:

MC-S-P
wherein:
M is a radiometal and C is a chelator which binds M;
S is an optional spacer covalently linked between C and the N-terminal of P;
P is a GRP receptor peptide antagonist of the general formula:

Xaa1-Xaa2-Xaa3-Xaa4-Xaa5-Xaa6-Xaa7-Z;
Xaa1 is not present or is selected from the group consisting of amino acid residues Asn, Thr, Phe, 3-(2-thienyl) alanine (Thi), 4-chlorophenylalanine (Cpa), α-naphthylalanine (α-Nal), β-naphthylalanine (β-Nal), 1,2,3,4-tetrahydronorharman-3-carboxylic acid (Tpi), Tyr, 3-iodo-tyrosine (o-I-Tyr), Trp and pentafluorophenylalanine (5-F-Phe) (all as L- or D-isomers);
Xaa2 is Gln, Asn or His;
Xaa3 is Trp or 1, 2, 3, 4-tetrahydronorharman-3-carboxylic acid (Tpi);
Xaa4 is Ala, Ser or Val;
Xaa5 is Val, Ser or Thr;
Xaa6 is Gly, sarcosine (Sar), D-Ala, or β-Ala;
Xaa7 is His or (3-methyl)histidine (3-Me)His;
Z is selected from —NHOH, —NHNH2, —NH-alkyl, —N(alkyl)2, and —O-alkyl
or Z is

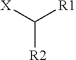

wherein X is NH (amide) or O (ester) and R1 and R2 are the same or different and selected from a proton, an optionally substituted alkyl, an optionally substituted alkyl ether, an aryl, an aryl ether or an alkyl-, halogen, hydroxyl or hydroxyalkyl substituted aryl or heteroaryl group; and
a surfactant comprising a compound having (i) a polyethylene glycol chain and (ii) a fatty acid ester.

In a second aspect, the present disclosure relates to a composition comprising a radiolabeled GRPR-antagonist for use in treating or preventing cancer in a subject, wherein
the radiolabeled GRPR-antagonist is of the following formula:

MC-S-P
wherein:
M is a radiometal and C is a chelator which binds M;
S is an optional spacer covalently linked between C and the N-terminal of P;
P is a GRP receptor peptide antagonist of the general formula:

Xaa1-Xaa2-Xaa3-Xaa4-Xaa5-Xaa6-Xaa7-Z;
Xaa1 is not present or is selected from the group consisting of amino acid residues Asn, Thr, Phe, 3-(2-thienyl) alanine (Thi), 4-chlorophenylalanine (Cpa), α-naphthylalanine (α-Nal), β-naphthylalanine (β-Nal), 1,2,3,4-tetrahydronorharman-3-carboxylic acid (Tpi), Tyr, 3-iodo-tyrosine (o-l-Tyr), Trp and pentafluorophenylalanine (5-F-Phe) (all as L- or D-isomers);
Xaa2 is Gln, Asn or His;
Xaa3 is Trp or 1, 2, 3, 4-tetrahydronorharman-3-carboxylic acid (Tpi);
Xaa4 is Ala, Ser or Val;
Xaa5 is Val, Ser or Thr;
Xaa6 is Gly, sarcosine (Sar), D-Ala, or β-Ala;
Xaa7 is His or (3-methyl)histidine (3-Me)His;
Z is selected from —NHOH, —NHNH2, —NH-alkyl, —N(alkyl)2, and —O-alkyl
or Z is

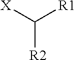

wherein X is NH (amide) or O (ester) and R1 and R2 are the same or different and selected from a proton, an optionally substituted alkyl, an optionally substituted alkyl ether, an aryl, an aryl ether or an alkyl-, halogen, hydroxyl or hydroxyalkyl substituted aryl or heteroaryl group; and
the radiolabeled GRPR-antagonist is administered to said subject at a therapeutically efficient amount comprised between 2000 and 10000 MBq.

### BRIEF DESCRIPTION OF THE DRAWINGS/FIGURES

FIG. 1A. FIG. 1A shows SPECT/CT images 4 hours and 24 hours post the 1st injection, and 4 hours post the 2nd and 3rd injection. Arrows indicate the tumor. Animals were either injected with 30 MBq/300 pmol (group 1), 40 MBq/400 pmol (group 2) or 60 MBq/600 pmol ¹⁷⁷Lu-NeoBOMB1.

FIG. 1B. FIG. 1B shows quantified tumor uptake (n=2 per group) from the injections described in FIG. 1A.

FIG. 2A,B. FIG. 2A shows extrapolated tumor size of untreated animals and animals treated with 3×30 MBq/300 pmol (group 1), 3×40 MBq/400 pmol (group 2) and 3×60 MBq/600 pmol ¹⁷⁷Lu-NeoBOMB1 (group 3). FIG. 2B shows survival of untreated animals and animals treated with 3×30 MBq/300 pmol (group 1), 3×40 MBq/400 pmol (group 2) and 3×60 MBq/600 pmol ¹⁷⁷Lu-NeoBOMB1 (group 3).

FIG. 3A,B. FIG. 3A shows animal weight before and after treatment up to 12 weeks after treatment. FIG. 3B shows animal weight before and after treatment up to 24 weeks after treatment.

FIG. 4. FIG. 4 shows representative hematoxylin and eosin staining of pancreatic tissue of untreated and treated animals (3×30 MBq/300 pmol (group 1), 3×40 MBq/400 pmol (group 2) and 3×60 MBq/600 pmol 177Lu-NeoBOMB1 (group 3)).

FIG. 5. FIG. 5 shows representative hematoxylin and eosin staining of kidney tissue of untreated and treated animals (3×30 MBq/300 pmol (group 1), 3×40 MBq/400 pmol (group 2) and 3×60 MBq/600 pmol ¹⁷⁷Lu-NeoBOMB1 (group 3)). Area's encircled indicate lesions with lymphocyte infiltration (ID: D, 814, 861, 868 and 862) or atrophy and fibrosis (ID: 864).

### DETAILED DESCRIPTION

### Definitions

The phrase “treatment of” and “treating” includes the amelioration or cessation of a disease, disorder, or a symptom thereof.

The phrase “prevention of” and “preventing” includes the avoidance of the onset of a disease, disorder, or a symptom thereof.

Consistent with the International System of Units, “MBq” is the abbreviation for the unit of radioactivity “megabecquerel.”

As used herein, “PET” stands for positron-emission tomography.

As used herein, “SPECT” stands for single-photon emission computed tomography.

As used herein, the terms “effective amount” or “therapeutically efficient amount” of a compound refer to an amount of the compound that will elicit the biological or medical response of a subject, for example, ameliorate the symptoms, alleviate conditions, slow or delay disease progression, or prevent a disease.

As used herein, the terms “substituted” or “optionally substituted” refers to a group which is optionally substituted with one or more substituents selected from: halogen, —OR′, —NR′R″, —SR′, —SiR′R″R′″, —OC(O)R′, —C(O)R′, —CO₂R′, —C(O)NR′R″, —OC(O)NR′R″, —NR″C(O)R′, —NR′—C(O)NR″R′″, —NR″C(O)OR′, —NR—C(NR′R″R″″)=NR″″, —NR—C(NR′R″)=NR″″—S(O)R′, —S(O)₂R′, —S(O)₂NR′R″, —NRSO₂R′, —CN, —NO₂, —R′, —N₃, —CH(Ph)₂, fluoro(C₁-C₄)alkoxo, and fluoro(C₁-C₄)alkyl, in a number ranging from zero to the total number of open valences on aromatic ring system; and where R′, R″, R′″ and R″″ may be independently selected from hydrogen, alkyl, heteroalkyl, cycloalkyl, heterocycloalkyl, aryl and heteroaryl. When a compound of the disclosure includes more than one R group, for example, each of the R groups is independently selected as are each R′, R″, R′″ and R″″ groups when more than one of these groups is present.

As used herein, the terms “alkyl”, by itself or as part of another substituent, refer to a linear or branched alkyl functional group having 1 to 12 carbon atoms. Suitable alkyl groups include methyl, ethyl, n-propyl, i-propyl, n-butyl, i-butyl, s-butyl and t-butyl, pentyl and its isomers (e.g. n-pentyl, iso-pentyl), and hexyl and its isomers (e.g. n-hexyl, iso-hexyl).

As used herein, the terms “heteroaryl” refer to a polyunsaturated, aromatic ring system having a single ring or multiple aromatic rings fused together or linked covalently, containing 5 to 10 atoms, wherein at least one ring is aromatic and at least one ring atom is a heteroatom selected from N, O and S. The nitrogen and sulfur heteroatoms may optionally be oxidized and the nitrogen heteroatoms may optionally be quaternized. Such rings may be fused to an aryl, cycloalkyl or heterocyclyl ring. Non-limiting examples of such heteroaryl, include: furanyl, thiophenyl, pyrrolyl, pyrazolyl, imidazolyl, oxazolyl, isoxazolyl, thiazolyl, isothiazolyl, triazolyl, oxadiazolyl, thiadiazolyl, tetrazolyl, oxatriazolyl, thiatriazolyl, pyridinyl, pyrimidyl, pyrazinyl, pyridazinyl, oxazinyl, dioxinyl, thiazinyl, triazinyl, indolyl, isoindolyl, benzofuranyl, isobenzofuranyl, benzothiophenyl, isobenzothiophenyl, indazolyl, benzimidazolyl, benzoxazolyl, purinyl, benzothiadiazolyl, quinolinyl, isoquinolinyl, cinnolinyl, quinazolinyl and quinoxalinyl.

As used herein, the terms “aryl” refer to a polyunsaturated, aromatic hydrocarbyl group having a single ring or multiple aromatic rings fused together, containing 6 to 10 ring atoms, wherein at least one ring is aromatic. The aromatic ring may optionally include one to two additional rings (cycloalkyl, heterocyclyl or heteroaryl as defined herein) fused thereto. Suitable aryl groups include phenyl, naphtyl and phenyl ring fused to a heterocyclyl, like benzopyranyl, benzodioxolyl, benzodioxanyl and the like.

As used herein, the term “halogen” refers to a fluoro (—F), chloro (—Cl), bromo (—Br), or iodo (—I) group

As used herein the terms “optionally substituted aliphatic chain” refers to an optionally substituted aliphatic chain having 4 to 36 carbon atoms, preferably 12 to 24 carbon atoms.

### Radiolabeled GRPR-Antagonist

As used herein, the GRPR-antagonist has the following formula:

MC-S-P
wherein:
M is a radiometal and C is a chelator which binds M;
S is an optional spacer covalently linked between C and the N-terminal of P;
P is a GRP receptor peptide antagonist of the general formula:

Xaa1-Xaa2-Xaa3-Xaa4-Xaa5-Xaa6-Xaa7-Z;
Xaa1 is not present or is selected from the group consisting of amino acid residues Asn, Thr, Phe, 3-(2-thienyl) alanine (Thi), 4-chlorophenylalanine (Cpa), α-naphthylalanine (α-Nal), β-naphthylalanine (β-Nal), 1,2,3,4-tetrahydronorharman-3-carboxylic acid (Tpi), Tyr, 3-iodo-tyrosine (o-I-Tyr), Trp and pentafluorophenylalanine (5-F-Phe) (all as L- or D-isomers);
Xaa2 is Gln, Asn or His;
Xaa3 is Trp or 1, 2, 3, 4-tetrahydronorharman-3-carboxylic acid (Tpi);
Xaa4 is Ala, Ser or Val;
Xaa5 is Val, Ser or Thr;
Xaa6 is Gly, sarcosine (Sar), D-Ala, or β-Ala;
Xaa7 is His or (3-methyl)histidine (3-Me)His;
Z is selected from —NHOH, —NHNH2, —NH-alkyl, —N(alkyl)2, and —O-alkyl
or Z is

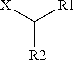

wherein X is NH (amide) or O (ester) and R1 and R2 are the same or different and selected from a proton, an optionally substituted alkyl, an optionally substituted alkyl ether, an aryl, an aryl ether or an alkyl-, halogen, hydroxyl or hydroxyalkyl substituted aryl or heteroaryl group.

According to an embodiment, Z is selected from one of the following formulae, wherein X is NH or O:

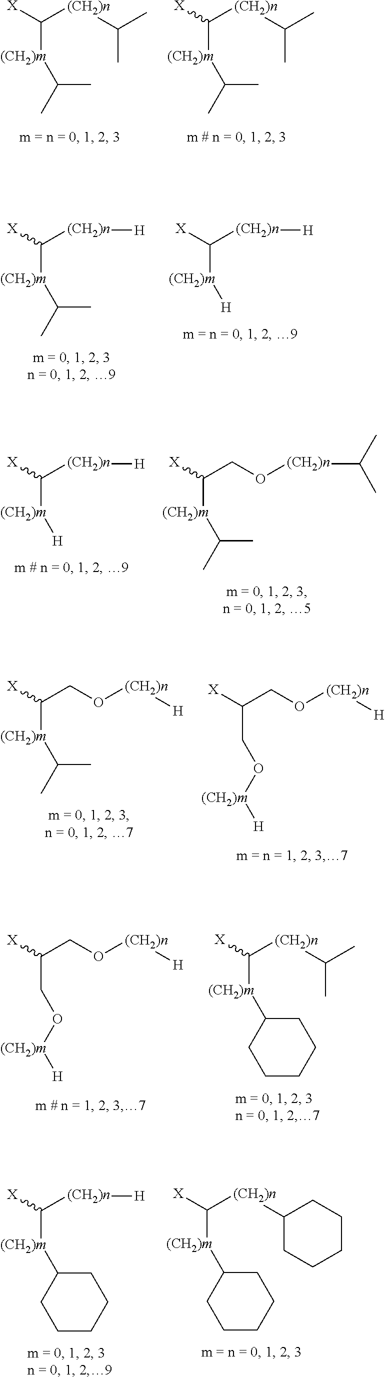

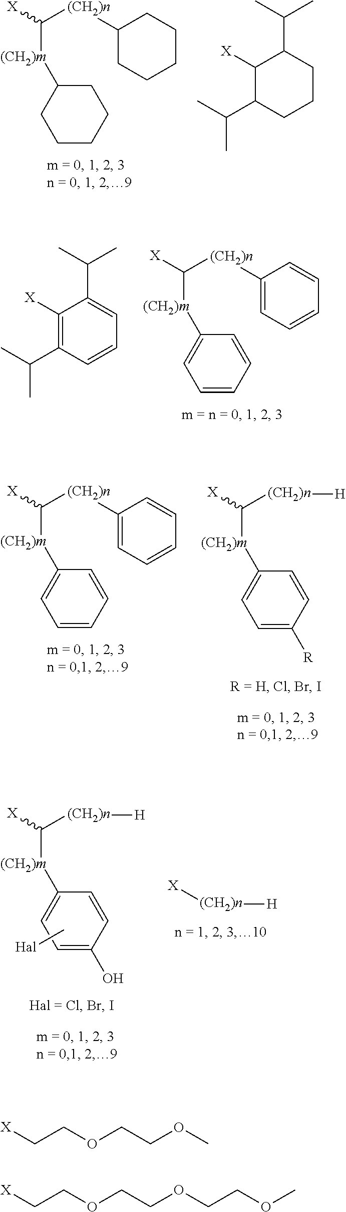

According to an embodiment, the chelator C is selected from the group consisting of:

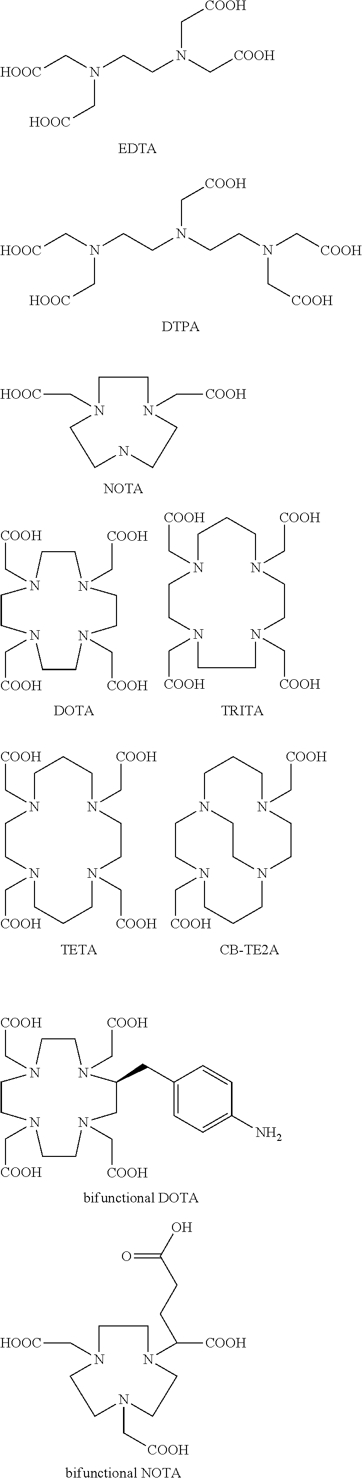

In specific embodiments, C is selected from the group consisting of:

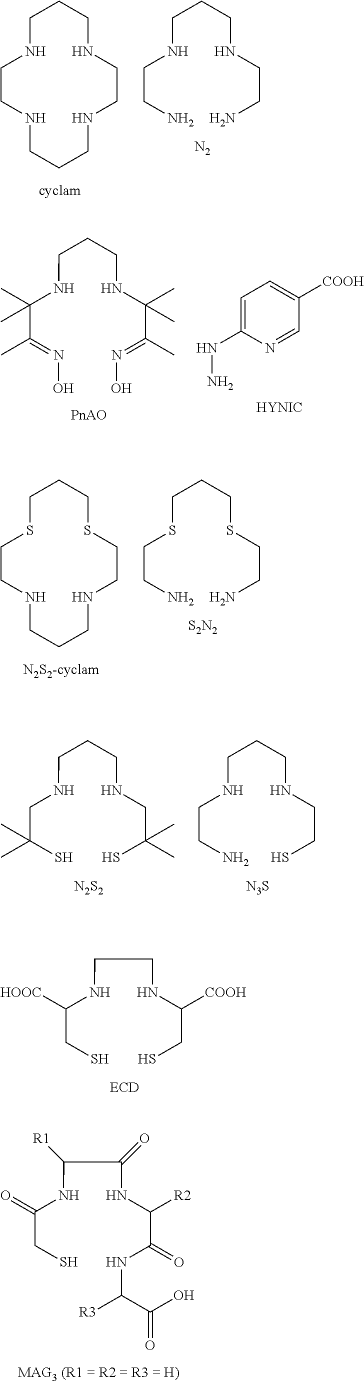

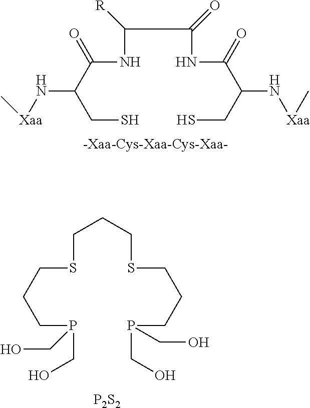

According to an embodiment, S is elected from S is selected from the group consisting of:

a) aryl containing residues of the formulae:

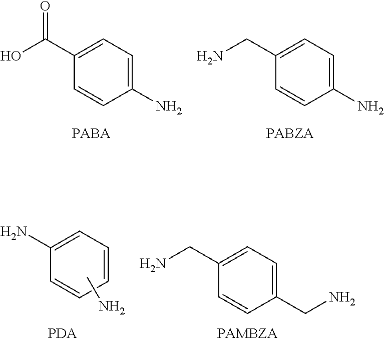

wherein PABA is p-aminobenzoic acid, PABZA is p-aminobenzylamine, PDA is phenylenediamine and PAMBZA is (aminomethyl) benzylamine;
b) dicarboxylic acids, ω-aminocarboxylic acids, ω-diaminocarboxylic acids or diamines of the formulae:

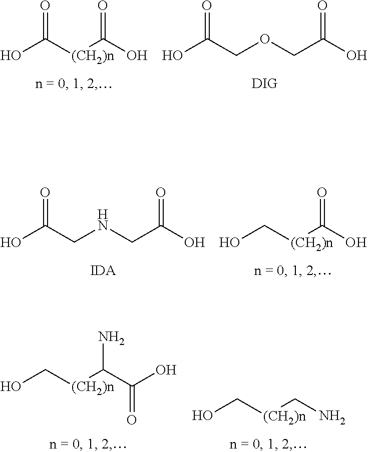

wherein DIG is diglycolic acid and IDA is iminodiacetic acid;
c) PEG spacers of various chain lengths, in particular PEG spacers sele

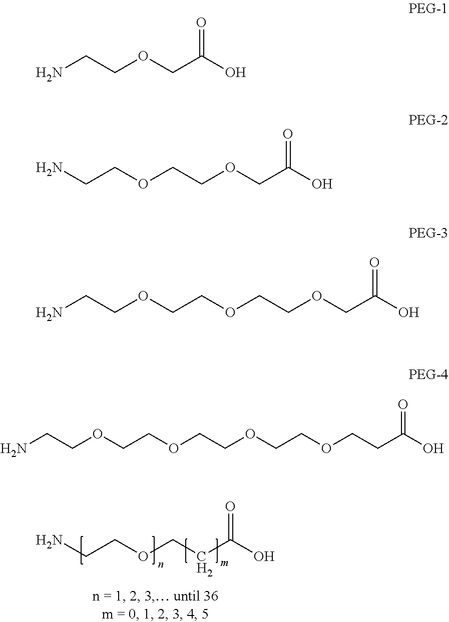

d) α- and β-amino acids, single or in homologous chains various chain lengths or heterologous chains of various chain lengths, in particular:

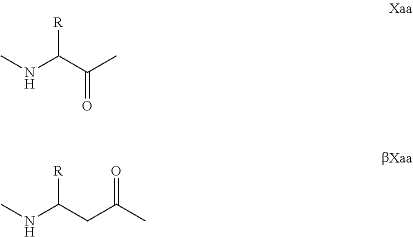

### GRP(1-18), GRP(14-18), GRP(13-18), BBN(1-5), or [Tyr4] BB (1-5); or

e) combinations of a, b, c and d.

According to an embodiment, the GRPR antagonist is selected from the group consisting of compounds of the following formulae:

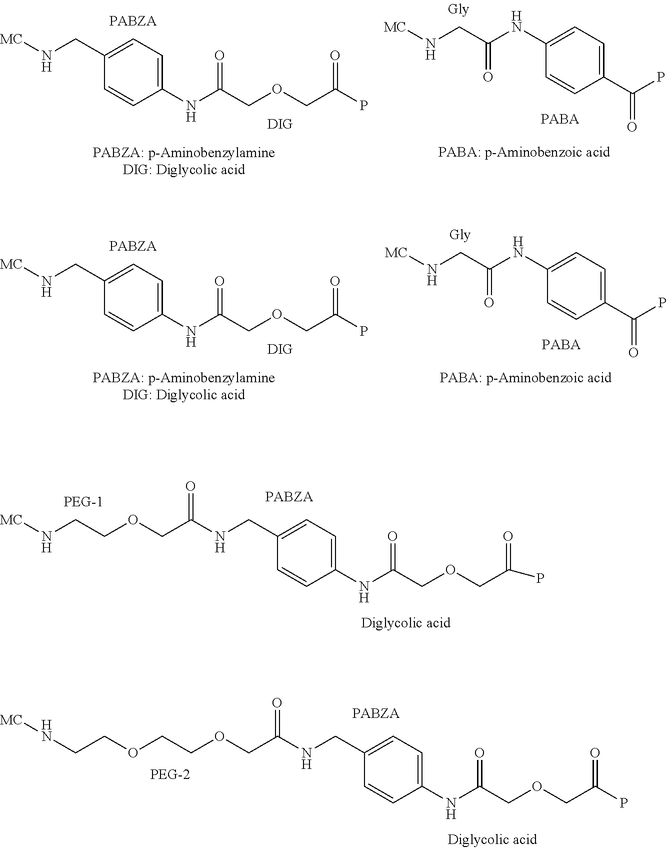

wherein MC and P are as defined above.

According to an embodiment P is DPhe-Gln-Trp-Ala-Val-Gly-His-NH—CH(CH₂—CH(CH₃)₂)₂.

According to an embodiment, the radiolabeled GRPR-antagonist is radiolabeled NeoBOMB1 of formula (I):

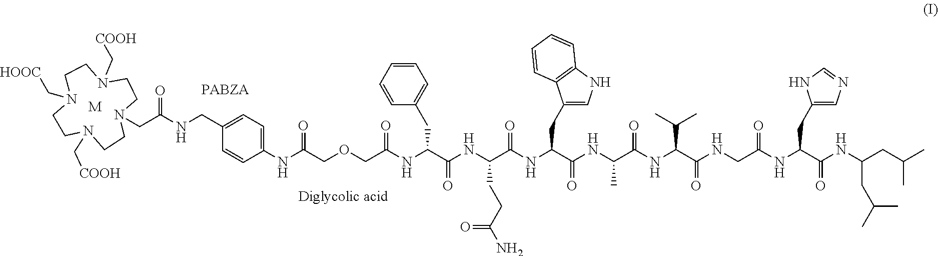

(M-DOTA-(p-aminobenzylamine-diglycolic acid)-[D-Phe⁶, His-NH—CH[(CH₂—CH(CH₃)₂]₂¹²,des-Leu¹³,des-Met¹⁴]BBN(6-14));
wherein M is a radiometal, preferably M is selected from ¹⁷⁷Lu, ⁶⁸Ga and ¹¹¹In.

According to an embodiment, the radiolabeled GRPR-antagonist is radiolabeled NeoBOMB2 of formula (II):

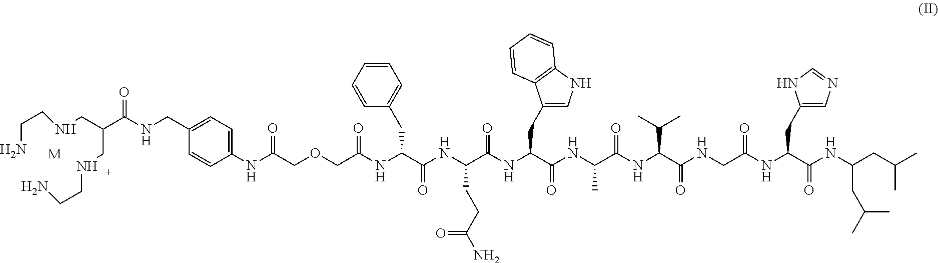

(M-N₄ (p-aminobenzylamine-diglycolic acid)-[D-Phe⁶, His-NH—CH[(CH₂—CH(CH₃)₂]₂¹²,des-Leu¹³,des-Met¹⁴]BBN(6-14));
wherein M is a radiometal.

In an embodiment, M is a radiometal which can be selected from selected from, ¹¹¹In, ^{33m}In, ⁹⁹mTc, ^{94m}Tc, ⁶⁷Ga ⁶Ga, ⁶⁸Ga ⁵²Fe, ¹⁶⁹Er, As, ⁹⁷Ru, ²⁰³Pb, ²¹²Pb, ⁶²Cu, ⁶⁴Cu, ⁶⁷Cu, ¹⁸⁶Re, ¹⁸⁸Re, ⁸⁶Y, ⁹⁰Y, ⁵¹Cr, ^{52m}Mn, ¹⁵⁷Gd, ¹⁷⁷Lu, ¹⁶¹Tb, ⁶⁹Yb, ¹⁷⁵Yb, ¹⁰⁵Rh, ¹⁶⁶Dy, ¹⁶⁶Ho, ¹⁵³Sm, ¹⁴⁹Pm, ¹⁵¹Pm, ¹⁷²Tm, ¹²¹Sn, ^{117m}Sn, ²¹³Bi, ²¹²Bi, ¹⁴²Pr, ¹⁴³Pr, ¹⁹⁸Au, ¹⁹⁹Au, ⁸⁹Zr, ²²⁵Ac and ⁴⁷Sc. Preferably M is selected from ¹⁷⁷Lu, ⁶⁸Ga and ¹¹¹In.

According to an embodiment, M is ¹⁷⁷Lu. In this case, the radiolabeled GRPR-antagonist could be used for radionuclide therapy. According to another embodiment, M is ⁶⁸Ga. In this case, the radiolabeled GRPR-antagonist could be used for PET. According to another embodiment, M is ¹¹¹In. In this case, the radiolabeled GRPR-antagonist could be used for SPECT.

### Pharmaceutical Composition

The GRPR-antagonist has the tendency to stick to glass and plastic surfaces due to non-specific binding (NSB), which is a problem for formulating the pharmaceutical composition. In order to provide a stable composition, several surfactants were tested. The inventors unexpectedly found that among all the tested surfactants, surfactants comprising a compound having (i) a polyethylene glycol chain and (ii) a fatty acid ester gave the best results.

In a first aspect, the present disclosure relates to a pharmaceutical composition comprising a radiolabeled GRPR-antagonist as described herein and a surfactant comprising a compound having (i) a polyethylene glycol chain and (ii) a fatty acid ester. In an embodiment, the surfactant also comprises free ethylene glycol.

In an embodiment, the surfactant comprises a compound of formula (III)

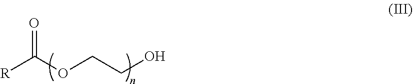

wherein n is comprised between 3 and 1000, preferably between 5 and 500, and more preferably between 10 and 50, and
R is the fatty acid chain, preferably an optionally substituted aliphatic chain.

In an embodiment, the surfactant comprises polyethylene glycol 15-hydroxystearate and free ethylene glycol.

The radiolabeled GRPR-antagonist can be present in a concentration providing a volumetric radioactivity of at least 100 MBq/mL, preferably at least 250 MBq/mL. The radiolabeled GRPR-antagonist can be present in a concentration providing a volumetric radioactivity comprised between 100 MBq/mL and 1000 MBq/mL, preferably between 250 MBq/mL and 500 MBq/mL.

The surfactant can be present in a concentration of at least 5 μg/mL, preferably at least 25 μg/mL, and more preferably at least 50 μg/mL. The surfactant can be present in a concentration comprised between 5 μg/mL and 5000 μg/mL, preferably between 25 μg/mL and 2000 μg/mL, and more preferably between 50 μg/mL and 1000 μg/mL.

In an embodiment, the composition comprises at least one other pharmaceutically acceptable excipient. The pharmaceutically acceptable excipient can be any of those conventionally used, and is limited only by physico-chemical considerations, such as solubility and lack of reactivity with the active compound(s).

In particular, the one or more excipient(s) can be selected from stabilizers against radiolytic degradation, buffers, sequestering agents and mixtures thereof.

As used herein, “stabilizer against radiolytic degradation” refers to stabilizing agent which protects organic molecules against radiolytic degradation, e.g. when a gamma ray emitted from the radionuclide is cleaving a bond between the atoms of an organic molecules and radicals are forms, those radicals are then scavenged by the stabilizer which avoids the radicals undergo any other chemical reactions which might lead to undesired, potentially ineffective or even toxic molecules. Therefore, those stabilizers are also referred to as “free radical scavengers” or in short “radical scavengers”. Other alternative terms for those stabilizers are “radiation stability enhancers”, “radiolytic stabilizers”, or simply “quenchers”.

As used herein, “sequestering agent” refers to a chelating agent suitable to complex free radionuclide metal ions in the formulation (which are not complexed with the radiolabelled peptide).

Buffers include acetate buffer, citrate buffer and phosphate buffer.

According to an embodiment the pharmaceutical composition is an aqueous solution, for example an injectable formulation. According to a particular embodiment, the pharmaceutical composition is a solution for infusion.

The requirements for effective pharmaceutical carriers for injectable compositions are well-known to those of ordinary skill in the art (see, e.g., Pharmaceutics and Pharmacy Practice, J.B. Lippincott Company, Philadelphia, Pa., Banker and Chalmers, eds., pages 238-250 (1982), and \hat{ }SHP Handbook on Injectable Drugs, Trissel, 15th ed., pages 622-630 (2009)).

The disclosure also relates to a method of manufacturing a pharmaceutical composition comprising combining a radiolabeled GRPR-antagonist and a surfactant.

The disclosure also relates to the pharmaceutical composition as described above for use in treating or preventing cancer.

As used herein, the terms “cancer” refer to cells having the capacity for autonomous growth, i.e., an abnormal state or condition characterized by rapidly proliferating cell growth. Hyperproliferative and neoplastic disease states may be categorized as pathologic, i.e., characterizing or constituting a disease state, or may be categorized as non-pathologic, i.e., a deviation from normal but not associated with a disease state. The term is meant to include all types of cancerous growths or oncogenic processes, metastatic tissues or malignantly transformed cells, tissues, or organs, irrespective of histopathologic type or stage of invasiveness.

In specific embodiments, the cancer is selected from prostate cancer, breast cancer, small cell lung cancer, colon carcinoma, gastrointestinal stromal tumors, gastrinoma, renal cell carcinomas, gastroenteropancreatic neuroendocrine tumors, oesophageal squamous cell tumors, neuroblastomas, head and neck squamous cell carcinomas, as well as ovarian, endometrial and pancreatic tumors displaying neoplasia-related vasculature that is GRPR. In an embodiment, the cancer is prostate cancer or breast cancer.

The disclosure also relates to a pharmaceutical composition according as described above for use in in vivo imaging, in particular for detecting GRPR positive tumors in a subject in need thereof, preferably by PET and SPECT imaging.

The disclosure also relates to a method for treating or preventing cancer in a subject in need thereof, the method comprising administering to said subject a therapeutically efficient amount of the pharmaceutical composition as described above.

The disclosure also relates to a method for in vivo imaging, the method comprising administering to a subject, an effective amount of the pharmaceutical composition as described above and detecting the signal derived from the decay of the radioisotope present in said compound.

### Radiolabeled GRPR-Antagonist for Use in Treating Cancer

In a second aspect, the disclosure also relates a composition comprising a radiolabeled GRPR-antagonist for use in treating or preventing cancer in a subject in need thereof, wherein the radiolabeled GRPR-antagonist is administered to said subject at a therapeutically efficient amount comprised between 2000 and 10000 MBq.

In specific embodiments, a therapeutically efficient amount of the composition is administered to said subject 2 to 8 times per treatment. For example, a patient may be treated with radiolabelled GRPR antagonist, specifically ¹⁴⁷Lu-NeoBOMB1, intravenously in 2 to 8 cycles of a 2000 to 10000 MBq each.

In certain aspects the subject is a mammal, for example but not limited to a rodent, canine, feline, or primate. In certain aspects, the subject is a human.

The inventors found out that ¹⁷⁷Lu-NeoBOMB1 is effective as shown in animal models of cancer. Compared to untreated animals, treatment groups had a significantly longer tumor growth delay time and a significantly longer median survival time. In the non-limiting Examples described herein, animals were either treated with 3×30 MBq/300 pmol, 3×40 MBq/400 pmol or 3×60 MBq/600 pmol ¹⁷⁷Lu-NeoBOMB1. No significant difference in tumor growth delay time and median survival were found between the treatment groups though. This finding was unexpected, as prior dosimetry calculations using the linear-quadratic model predicted a difference in tumor control probability between the treatment groups (tumor control probability: 0%, 75% and 100%, for animals treated with 3×30 MBq/300 pmol, 3×40 MBq/400 pmol and 3×60 MBq/600 pmol, respectively). Without being bound by any theory, it is predicted that a dose necessary to treat a patient would be much lower than expected from the prior dosimetry calculations, which would lead to a lower toxicity of the radiolabeled NeoBOMB1.

Advantageously, the radiolabeled GRPR-antagonist is labeled with ¹⁷⁷Lu.

In specific embodiments of the above methods, the cancer is selected from prostate cancer, breast cancer, small cell lung cancer, colon carcinoma, gastrointestinal stromal tumors, gastrinoma, renal cell carcinomas, gastroenteropancreatic neuroendocrine tumors, oesophageal squamous cell tumors, neuroblastomas, head and neck squamous cell carcinomas, as well as ovarian, endometrial and pancreatic tumors displaying neoplasia-related vasculature that are GRPR positive. In an embodiment, the cancer is prostate cancer or breast cancer.

According to an embodiment, the composition for use is the pharmaceutical composition as described in the previous section.

The disclosure also relates to a method of treating or preventing a cancer, the method comprising administering to a subject with cancer an effective amount of a composition comprising radiolabeled GRPR-antagonist, wherein the radiolabeled GRPR-antagonist is administered to said subject at a therapeutically efficient amount comprised between 2000 and 10000 MBq.

Provided herein is a method of treating or preventing a cancer, the method comprising administering to a subject with cancer an effective amount of a composition comprising radiolabeled GRPR-antagonist as disclosed herein. In certain aspects, the cancer is prostate cancer or breast cancer.

In certain aspects, the administration of the composition comprising radiolabeled GRPR-antagonist to a subject with cancer can inhibit, delay, and/or reduce tumor growth in the subject. In certain aspects, the growth of the tumor is delayed by at least 50%, 60%, 70% or 80% in comparison to an untreated control subject. In certain aspects, the growth of the tumor is delayed by at least 80% in comparison to an untreated control subject. In certain aspects, the growth of the tumor is delayed by at least 50%, 60%, 70% or 80% in comparison to the predicted growth of the tumor without the treatment. In certain aspects, the growth of the tumor is delayed by at least 80% in comparison to the predicted growth of the tumor without the treatment. One of ordinary skill in the art would recognize that predictions in tumor growth rate can be made based on epidemiological data, reports in medical literature and other knowledge in the field, the type of tumor and measurements of the tumor size, etc.

In certain aspects, the administration of the composition comprising radiolabeled GRPR-antagonist to a subject with cancer can increase the length of survival of the subject. In certain aspects, the increase in survival is in comparison to an untreated control subject. In certain aspects, the increase in survival is in comparison to the predicted length of survival of the subject without the treatment. In certain aspects, the length of survival is increased by at least 3 times, 4 times, or 5 times the length in comparison to an untreated control subject. In certain aspects, the length of survival is increased by at least 4 times the length in comparison to an untreated control subject. In certain aspects, the length of survival is increased by at least 3 times, 4 times, or 5 times the length in comparison to the predicted length of survival of the subject without the treatment. In certain aspects, the length of survival is increased by at least 4 times the length in comparison to the predicted length of survival of the subject without the treatment. In certain aspects, the length of survival is increased by at least one week, two weeks, one month, two months, three months, six months, one year, two years, or three years in comparison to an untreated control subject. In certain aspects, the length of survival is increased by at least one month, two months, or three months in comparison to an untreated control subject. In certain aspects, the length of survival is increased by at least one week, two weeks, one month, two months, three months, six months, one year, two years, or three years in comparison to the predicted length of survival of the subject without the treatment. In certain aspects, the length of survival is increased by at least one month, two months, or three months in comparison to the predicted length of survival of the subject without the treatment.

In certain aspects, the amount of radiolabeled GRPR-antagonist administered is less than the amount predicted for a subject to have 100% tumor control probability in the subject.

In certain aspects, the amount of radiolabeled GRPR-antagonist administered is less than the amount predicted for a subject to have at least 75% tumor control probability in the subject. In certain aspects, the amount of radiolabeled GRPR-antagonist administered is less than the amount predicted for a subject to achieve 50% tumor control probability in the subject. In certain aspects, the amount of radiolabeled GRPR-antagonist administered is less than the amount predicted for a subject to achieve 25% tumor control probability in the subject. In certain aspects, the amount of radiolabeled GRPR-antagonist administered is less than the amount predicted for a subject to achieve 10% tumor control probability in the subject. In certain aspects, the amount of radiolabeled GRPR-antagonist administered is not more than 25%, 30%, 40%, 50%, 60%, 70%, or 75% of the amount predicted for a subject to have 100% tumor control probability in the subject. In certain aspects, the amount of radiolabeled GRPR-antagonist administered is not more than 50%, 60%, 70%, 75%, 80%, or 85% of the amount predicted for a subject to have at least 75% tumor control probability in the subject. In certain aspects, the amount of radiolabeled GRPR-antagonist administered is not more than 60%, 65%, 70%, 75%, 80%, 85%, or 90% of the amount predicted for a subject to have at least 50% tumor control probability in the subject. In certain aspects, the amount of radiolabeled GRPR-antagonist administered is an amount predicted for a subject to have less than 25%, 20%, 15% 10%, or 5% tumor control probability. In certain aspects, the amount of radiolabeled GRPR-antagonist administered is an amount predicted for a subject to have 0% tumor control probability. In certain aspects, the amount of radiolabeled GRPR-antagonist administered is an amount predicted for a subject to have 0% tumor control probability.

### EXAMPLES

### Example 1: Screening of a Formulation for Reducing Adhesion of NeoBOMB1 Using ⁶⁸Ga-NeoBOMB1

During the development of the formulation kit, we realized that the peptide has a particular tendency to stick on glass and plastic surfaces.

This phenomenon is called Non specific binding (NSB). Peptides often demonstrate greater NSB issues than small molecules, especially uncharged peptides can adsorb strongly to plastics. The causes may be different: Physical/chemical properties, Van der Waals interactions, ionic interactions.

Organic solvent may enhance solubility and prevent adsorption. Ethanol for example can be used in radiopharmaceutical injections to enhance the solubility of highly lipophilic tracers or to decrease adsorption to vials, membrane filters, and injection syringes. We discarded the ethanol because it is not compatible with the freeze-drying.

Human Serum Albumin (HSA) is also used in a number of protein formulations as a stabilizer to prevent surface adsorption but this excipient in not suitable due to his thermal instability. Another possible approach was the use of surfactants (e.g Polysorbate 20, Polysorbate 80, Pluronic F-68, Sorbitan trioleate).

We focused our attention on the study of non-ionic surfactants, because the ionic surfactants may interfere in the labeling of ⁶⁸Ga.

Non-ionic tensioactives, like Kolliphor HS 15, Kolliphor K188, Tween 20, Tween 80, Polyvinylpyrrolidone K10, are commercially available as solubilizing excipients in oral and injectable formulations. In the table below are summarized the initial tests that have been done with different tensioactive agents.

### Materials and Method:

Labeling of NeoBOMB1 was based on a previously published kit approach by Castaldi et al. (Castaldi E, Muzio V, D'Angeli L, Fugazza L. ⁶⁸GaDOTATATE lyophilized ready to use kit for PET imaging in pancreatic cancer murine model, J Nucl Med 2014; 55(suppl 1):1926).

Different surfactants were screened and the adhesion % of the resulting aqueous solution was determined evaluating by dose calibrator measurement the total radioactivity left in the vial after a complete withdrawal of the radiolabeled solution. The difference, expressed as a percentage, between the total radioactivity measured before and after the sample withdrawal is directly correlated with the adhesion of the peptide on the container closure system. The results are summarized in Table 1.

TABLE 1
Tensioactive agents - effect on adhesion

| [symbol] | [symbol] | [symbol] |
|---|---|---|
| 50 | No tensioactive- | 21.4 |
| 50 | PEG 300 (500 μg) | 15.0 |
| 50 | EtOH 80% | 7.8 |
| 50 | DMSO 15% | 10.4 |
| 50 | ACN 80% | 7.9 |
| 50 | PEG 400 (500 μg) | 15.1 |
| 50 | PVP K10 (500 μg) | 15.6 |
| 50 | Albumin (500 μg) | 7.9 |
| 50 | Kolliphor P188 (1500 μg) | 7.9 |
| 50 | Hydroxy Propyl β Cyclodextrin (5000 μg) | 13.5 |
| 50 | PEG 4000 (500 μg) | 12.8 |
| 50 | Tween 20 (500 μg) | 6.1 |
| 50 | Kolliphor HS 15 (500 μg) | 6.2 |

[symbol]

indicates data missing or illegible when filed

The best results in term of peptide adhesion were obtained with the Kolliphor HS 15 and with Tween 20. The two excipients were further investigated to determine the final amount into the kit. The results obtained were good in term of radiochemical purity and peptide adhesion.

TABLE 2
Comparison between Tween 20 and Kolliphor HS 15

| [symbol] | [symbol] | [symbol] | [symbol] |
|---|---|---|---|
| 50 | Tween 20 (1.5 mg) | 94.3 | 5.2 |
| 50 | Tween 20 (0.5 mg) | 94.7 | 6.0 |
| 50 | Tween 20 (0.1 mg) | 95.3 | 7.6 |
| 50 | Kolliphor HS 15 (2 mg) | 92.8 | 5.9 |
| 50 | Kolliphor HS 15 (1.5 mg) | 93.4 | 4.9 |
| 50 | Kolliphor HS 15 (1.0 mg) | 94.3 | 4.6 |
| 50 | Kolliphor HS 15 (0.5 mg) | 94.9 | 4.2 |
| 50 | Kolliphor HS 15 (0.25 mg) | 95.1 | 6.0 |
| 50 | Kolliphor HS 15 (0.1 mg) | 96.1 | 8.3 |
| 50 | Kolliphor HS 15 (0.05 mg) | 95.7 | 9.0 |

[symbol]

indicates data missing or illegible when filed

We focused on Kolliphor HS 15 because the polysorbates (tween 20) may undergo autooxidation, cleavage at the ethylene oxide subunits and hydrolysis of the fatty acid ester bond caused by presence of oxygen, metal ions, peroxides or elevated temperature.

### Example 2: Preclinical Studies of the Therapeutic Efficacy of ⁷⁷Lu-NeoBOMB1

Disclosed herein are exemplary, non-limiting examples of preclinical studies of the therapeutic efficacy of ¹⁷⁷Lu-NeoBOMB1 involving treatment of animals xenografted with the well-known GRPR-expressing prostate cancer cell line PC-3 with 3 different doses of ¹⁷⁷Lu-NeoBOMB1. In addition, in a small group of non-tumor bearing animals the effect of ¹⁷⁷Lu-NeoBOMB1 treatment on kidneys and pancreas was studied by histopathological examination after treatment.

### Materials and Methods

### Radiolabeling

NeoBOMB1 (ADVANCED ACCELERATOR APPLICATIONS) (WO2014052471) was diluted in ultra-pure water, and concentration and chemical purity were monitored with an in-house-developed titration method (Breeman W A, de Zanger R M, Chan H S, de Blois E. Alternative method to determine specific activity of ¹⁷⁷Lu by HPLC. Curr Radiopharm. 2015; 8:119-122). Radioactivity was added (100 MBq/nmol ¹⁷⁷Lu) to a vial containing all the necessary excipients, for example, buffer, antioxidants, and peptide, including the tensioactive agent (Kolliphor HS15) to prevent sticking of the peptide. High-performance liquid chromatography was performed with a gradient of methanol and 0.1% trifluoroacetic acid to determine radiochemical purity. Radiometal incorporation measured by instant thin-layer chromatography on silica gel as previously described (de Blois E, Chan H S, Konijnenberg M, de Zanger R, Breeman W A. Effectiveness of quenchers to reduce radiolysis of (111)In- or (¹⁷⁷)Lu-labelled methionine-containing regulatory peptides. Maintaining radiochemical purity as measured by HPLC. Curr Top Med Chem. 2012; 12:2677-2685), was >67% and >90% for SPECT/CT, and efficacy and toxicity studies, respectively.

### Animal Model, Efficacy and Toxicity

All animal studies were conducted in agreement with the Animal Welfare Committee requirements of the Erasmus Medical Center and in accordance with accepted guidelines.

Male balb c nu/nu mice were subcutaneously inoculated on the right shoulder with 200 μL 4×10⁶ PC-3 cells (American Type Culture Collection) in inoculation medium (⅓ Matrigel high concentration (Corning)+⅔ Hank's balanced salt solution (Thermofisher Scientific)). Four weeks post tumor cell inoculation when an average tumor size of 543+177 mm³ was reached, animals were divided in four groups: control group (n=10) and therapy group 1-3 (n=15 per group). To determine the efficacy of ¹⁷⁷Lu-NeoBOMB1, animals received either 3 sham injections (control group), 3×30 MBq/300 pmol ¹⁷⁷Lu-NeoBOMB1 (group 1), 3×40 MBq/400 pmol ¹⁷⁷Lu-NeoBOMB1 (group 2) or 3×60 MBq/600 pmol ¹⁷⁷Lu-NeoBOMB1 (group 3) under isoflurane/O₂ anesthesia. Injections were administered intravenously and injections were given 1 week apart.

To determine the effect of treatment on pancreas and kidney tissue, non-tumor bearing balb c nu/nu male mice received the same treatment as the animals included in the efficacy study. At 2 different time points after the last therapeutic injection (12 weeks and 24 weeks p.i.) animals were euthanized and pancreas and kidney tissue was collected for pathological analysis.

In both studies, animal weight and/or tumor size was measured bi-weekly. When tumor size was ≥2000 mm³ or a decrease in animal weight ≥20% within 48 hours was observed, animals were removed from the study. In the efficacy study, animals were followed until the maximum allowed age of 230 days was reached.

### SPECT/CT

To quantify tumor uptake SPECT/CT imaging was performed in an additional group of PC-3 xenografted animals (n=2 per group). When tumor size was 477±57 mm³ animals were injected with the same peptide amounts as the animals included in the efficacy and the toxicity study. Four hours and 24 h post the 1st therapeutic injection, and 4 hours post the 2nd and 3rd therapeutic injection, whole-body SPECT/CT scans were performed on a hybrid SPECT/CT scanner (VECTor5, MILabs, Utrecht, The Netherlands). SPECT was performed in 30 minutes with 40 bed positions, using a 2.0-mm pinhole collimator with a reported spatial resolution of 0.85 mm (Ivashchenko O, van der Have F, Goorden M C, Ramakers R M, Beekman F J. Ultra-high-sensitivity submillimeter mouse SPECT. J Nucl Med. 2015; 56:470-475). SPECT images were reconstructed using photopeak windows of 113 and 208 keV, with a background window on either side of the photopeak with a width of 20% of the corresponding photopeak, and a SR-OSEM reconstruction method (Vaissier P E, Beekman F J, Goorden M C. Similarity-regulation of OS-EM for accelerated SPECT reconstruction. Phys Med Biol. 2016; 61:4300-4315), a voxel size of 0.8 mm³, and registered to the CT data. A post-reconstruction 3-dimensional Gaussian filter was applied (1 mm fwhm). CT was performed with the following settings: 0.24 mA, 50 kV, full angle scan, 1 position. The CT was reconstructed at 100 μm³.

### Pathological Analysis

Pancreatic and kidney tissue collected for pathological analysis was formalin fixed and paraffin embedded. Hematoxylin and eosin staining was performed on 4 pM thick tissue slices using the Ventana Symphony™ H&E protocol (Ventana), to determine differences in tissue structure between the 4 treatment groups. In total 4 tissue slices, 50 pM apart from each other were evaluated of each organ. The hematoxylin and eosin staining's were evaluated by experienced pathologists.

### Dosimetry

The RADAR realistic mouse model (Keenan M A, Stabin M G, Segars W P, Fernald M J. RADAR realistic animal model series for dose assessment. J Nucl Med. 2010; 51:471-476) with a weight of 25 g and data from previously published biodistribution and pharmacokinetic studies (Dalm S U, Bakker I L, de Blois E, et al. 68Ga/¹⁷⁷Lu-NeoBOMB1, a Novel Radiolabeled GRPR Antagonist for Theranostic Use in Oncology. J Nucl Med. 2017; 58:293-299) were used to calculate the dose to the tumor, pancreas and kidneys, when animals were treated with 3×30 MBq/300 pmol, 4×40 MBq/400 pmol or 3×60 MBq/600 pmol ¹⁷⁷Lu-NeoBOMB1. The biodistribution data of our previously published paper (Dalm S U, Bakker I L, de Blois E, et al. ⁶⁸Ga/¹⁷⁷Lu-NeoBOMB1, a Novel Radiolabeled GRPR Antagonist for Theranostic Use in Oncology. J Nucl Med. 2017; 58:293-299) were fitted with exponential curves to define the time-activity curve in the tumor and organs. The time integrated activities for ¹⁷⁷Lu were obtained by integrating these exponential curves folded with the 177Lu decay curve (T_{1/2}=6.647 d). Absorbed doses per administered activities were obtained by multiplying with the organ S-values from Keenan et al. (Keenan M A, Stabin M G, Segars W P, Fernald M J. RADAR realistic animal model series for dose assessment. J Nucl Med. 2010; 51:471-476) or by using the spherical node S-values (Stabin M G, Konijnenberg M W. Re-evaluation of absorbed fractions for photons and electrons in spheres of various sizes. J Nucl Med. 2000; 41:149-160) for a 340 mg tumor.

The tumor dosimetry was used for a prediction of the therapeutic outcome by using the Linear Quadratic (LQ) model based tumor control probability (TCP) (Konijnenberg M W, Breeman W A, de Blois E, et al. Therapeutic application of CCK2R-targeting PP-F11: influence of particle range, activity and peptide amount. EJNMMI Res. 2014; 4:47).

TCP=exp(−N_{clonogens}×S(D,T))

With N_{clonogens} the number of clonogenic (stem) cells within the tumor and S(D,T) the surviving fractions of cells as a function of absorbed dose D and time T. The LQ model indicates the survival as a function of absorbed dose for a tumor growing with a doubling time T_{d} by:

$$S ( D , T ) = exp [ - α D ( T ) ( 1 + \frac{G}{\frac{α}{β}} \times \frac{D ( T )}{N} ) + \frac{ln ( 2 )}{{T}_{d}} \times T ]$$

with α the radiation sensitivity of the tumor, α/β the ratio between the direct (α) and indirect (β) radiation sensitivity and G the time factor expressing the build-up of indirect damage during the dose delivery depending on the effective decay half-life and the half-life of sub-lethal damage repair. The tumor doubling times were determined by fitting an exponential growth function to the tumor volume over time in the control group. The radiation sensitivity parameters for PC-3 tumors were obtained from LDR and HDR brachytherapy survival data α=0.145 Gy and α/β=4.1 (2.5-5.7) Gy (Carlson D J, Stewart R D, Li X A, Jennings K, Wang J Z, Guerrero M. Comparison of in vitro and in vivo alpha/beta ratios for prostate cancer. Phys Med Biol. 2004; 49:4477-4491). The sub-lethal damage repair half-life for PC-3 tumors was indicated to be: 6.6 (5.3-8.0) h (Carlson D J, Stewart R D, Li X A, Jennings K, Wang J Z, Guerrero M. Comparison of in vitro and in vivo alpha/beta ratios for prostate cancer. Phys Med Biol. 2004; 49:4477-4491), but the value was conservatively fixed at a lower value of 1 h (Joiner M, Kogel Avd. Basic clinical radiobiology. 4th ed. London: Hodder Arnold; 2009). The TCP model was used to select administered activities that will lead to growth delay only (TCP=0%), partial response (TCP>75%) and complete response (TCP=100%). The clonogenic cell density in the PC-3 tumor xenografts was assumed to be 10⁶ cells/cm³.

### Tumor Volume Analysis

The tumor doubling times were determined by fitting an exponential growth function to the tumor volume over time in the control group. In the therapy groups an interval with exponential tumor volume decline was fitted with onset of regrowth after the nadir time. The growth curves were extrapolated beyond the censoring time points for mice with too large tumors (>2000 mm³) to determine average growth statistics. Tumor growth delay times were individually determined by comparing the times needed to reach the maximum tumor size of 2000 mm³ with the mean time found in the control group.

### Statistics

Prism software (version 5.01, GraphPad Software) was used for statistical analyses. P values >0.05 were considered statistically significant. The difference in tumor volume growths and delay times for the 4 groups were analyzed with the One-way ANOVA test with Bonferroni's multiple comparison test. Curve fitting was performed according to the least/square fit with the Pearson R² to quantify its goodness.

### Results

### SPECT/CT

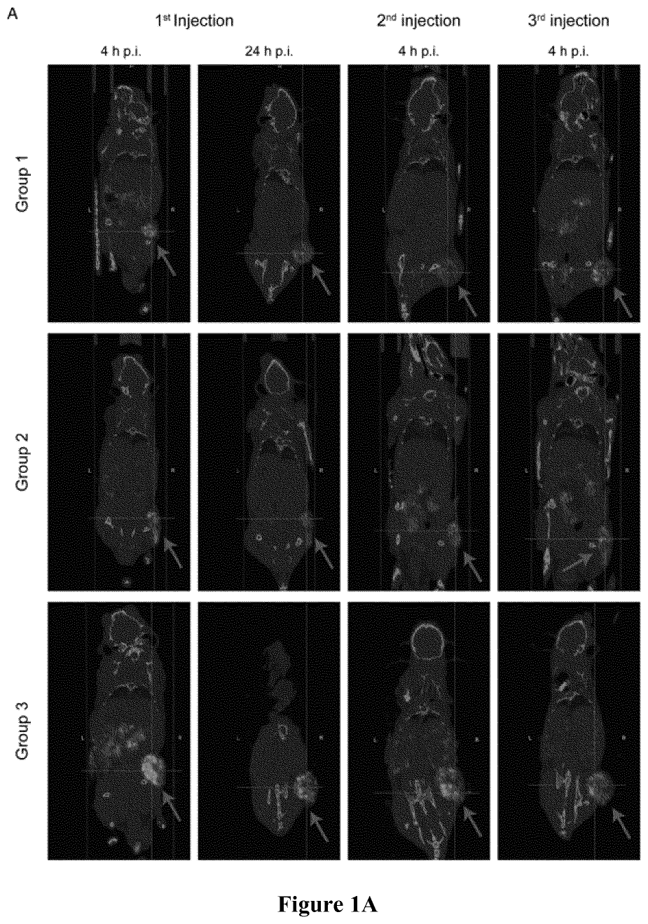

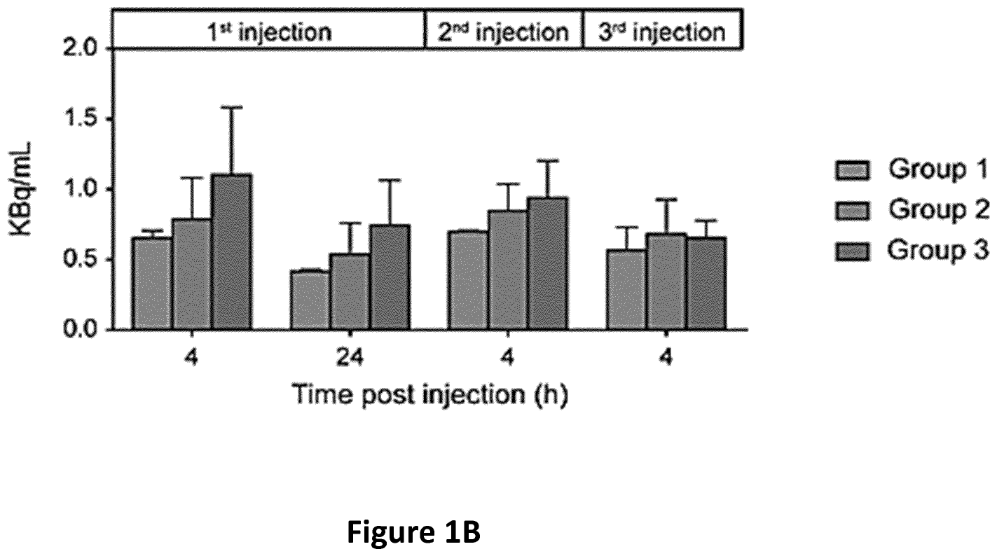

At most time points the average radioactivity uptake quantified on SPECT/CT was highest for group 3, followed by group 2 and group 1. However, the differences between the groups were not significant. FIG. 1A shows the scans of one animal of each group obtained at 4 hours and 24 hours after the 1st injection and 4 hours after the 2nd and 3rd injection. The quantified tumor uptake is depicted in FIG. 1B.

### ⁷⁷Lu-NeoBOMB1 Treatment Efficacy

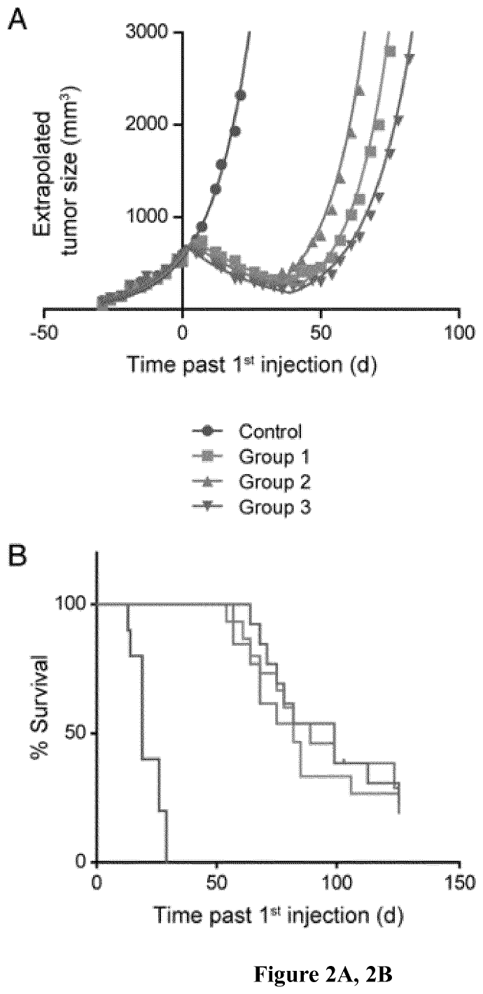

Therapy with ¹⁷⁷Lu-NeoBOMB1 proved to be effective. The animals in the control group reached a tumor size of 2000 mm³ within 20.3±5.9 d, while this was 97±59 d, 103±66 d and 95±26 d for group 1, group 2, and group 3, respectively (FIG. 2A). In addition, two animals from group 1 and one animal of group 2 did not show any tumor regrowth after a complete response. However, there was no significant difference in tumor growth delay times within the treatment groups, whereas the difference with the control group was highly significant (P<0.0001).

In line with the above, animals in the treatment groups had a significantly better survival compared to the treatment groups (P<0.001) (FIG. 2B). Median survival was 19 d, 82 d, 89 d and 99 d for the control group, group 1, group 2, and group 3, respectively.

Five animals (n=3 from group 2 and n=2 from group 3) were excluded from the study because of the following reasons; 1 animal was found death after the 1st injection, 1 animal had a very small tumor at the start of therapy that disappeared within a few days, 1 animal had more than 10% weight loss within 48 h and 1 animal retained fluids in the abdominal area. There was no sign that any of the mentioned events were related to treatment.

### Kidney and Pancreas Toxicity

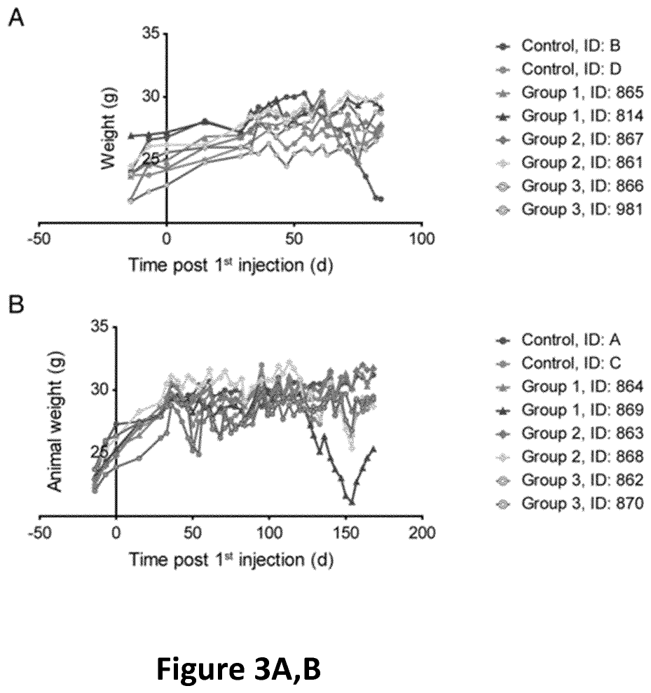

The animals included in the toxicity showed no critical decrease in weight throughout the follow-up period (FIG. 3). Animal weight increased in the first weeks and stayed relatively stable over time. One animal in the control group (ID: B) and one animal from Group 1 (ID: 869) showed a decrease in weight but this was less than 10% within 48 h.

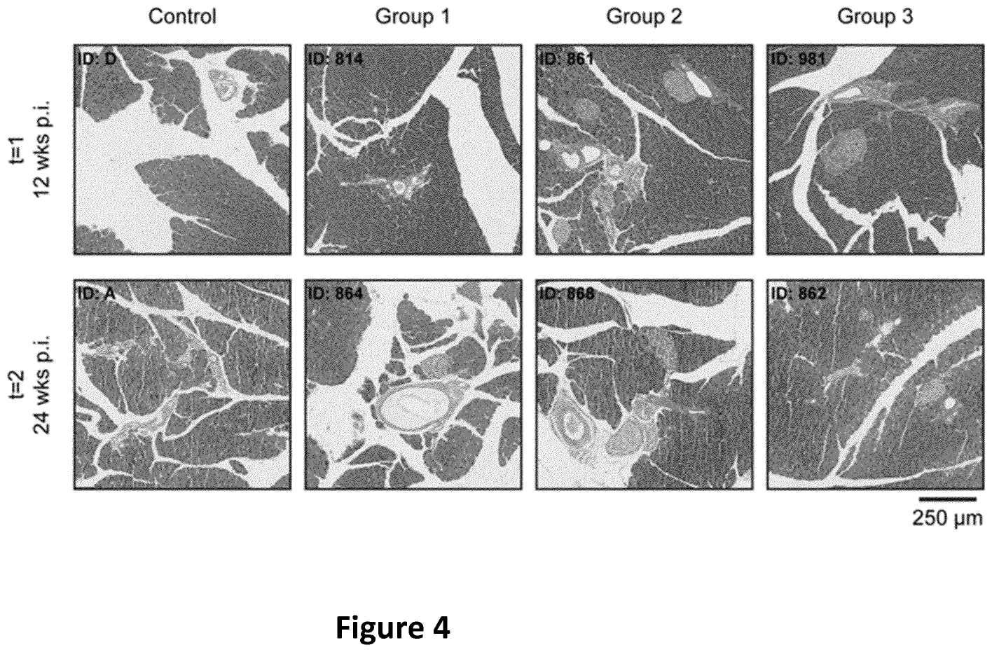

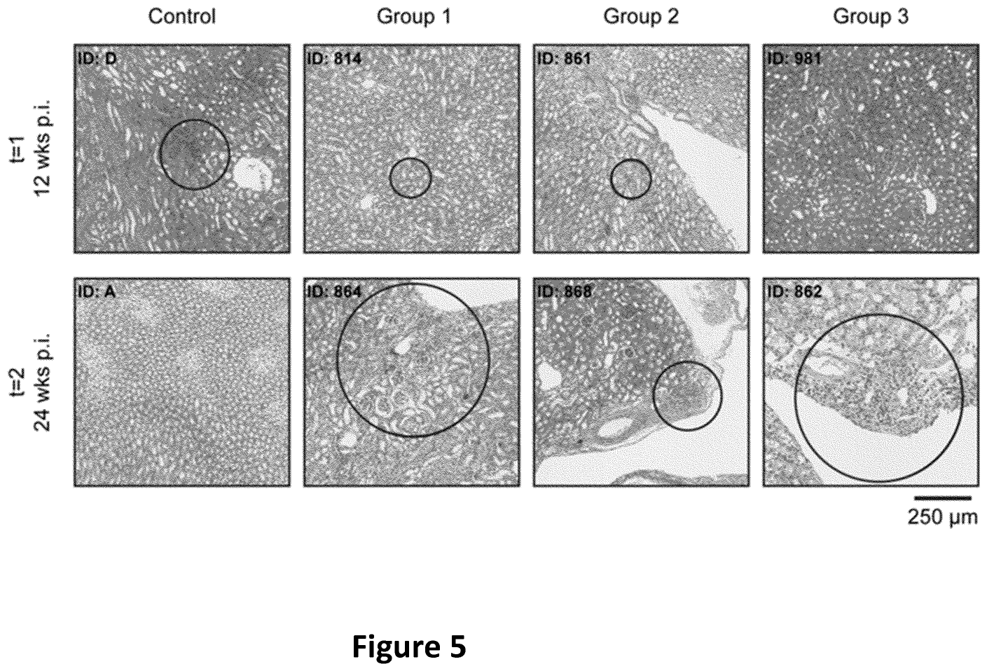

Histopathological analyses of the pancreas showed no tissue damage or other abnormalities (FIG. 4). Concerning the kidneys (FIG. 5), small areas with lymphocyte infiltration were observed in the kidneys at 12 weeks and 24 weeks after the last therapeutic injection. This was the case for the kidneys of control animals as well as treated animals, indicating that this finding was not related to therapy. Twenty-four weeks after therapy, atrophy and fibrosis were observed in the kidneys of only one animal that received the lowest therapeutic dose (ID: 864), unlikely to be related to therapy. In the kidneys of both animals from group 3 that were euthanized 24 weeks after therapy, a mild chronic inflammatory response was observed.

### Dosimetry

An estimation was made of the radioactivity dose to the tumor, pancreas and kidneys after treatment with 3×30 MBq/300 pmol, 3×40 MBq/400 pmol or 3×60 MBq/600 pmol ¹⁷⁷Lu-NeoBOMB1 (See Table 3 below). For this it was assumed that tumor and organ uptake was similar after each injection.

TABLE 3
Estimated doses to the tumor, pancreas and kidney when animals are
treated with with 3 × 30 MBq/300 pmol, 3 × 40 MBq/400 pmol or
3 × 60 MBq/600 pmol ¹⁷⁷Lu-NeoBOMB1*
Cumulative absorbed dose (Gy)

| | Injected dose | | | | | | | | |
|---|---|---|---|---|---|---|---|---|---|
| | 3 × 30 MBq/ | | | 3 × 40 MBq/ | | | 3 × 60 MBq/ | | |
| | 300 pmol | | | 400 pmol | | | 600 pmol | | |
| Injection | 1^{st} | 2^{nd} | 3^{rd} | 1^{st} | 2^{nd} | 3^{rd} | 1^{st} | 2^{nd} | 3^{rd} |
| Tumor | 17 | 34 | 68 | 23 | 46 | 91 | 34 | 68 | 137 |
| Kidney | 1.7 | 3.4 | 5.1 | 2.3 | 4.5 | 6.8 | 3.4 | 6.8 | 10 |
| Pancreas | 7.9 | 16 | 32 | 11 | 21 | 42 | 16 | 32 | 64 |

1-14. (canceled)

15. A pharmaceutical composition comprising
a radiolabeled GRPR-antagonist of the following formula:
MC-S-P
wherein:
M is a radiometal and C is a chelator which binds M;
S is an optional spacer covalently linked between C and the N-terminal of P;
P is a GRP receptor peptide antagonist of the general formula:
Xaa1-Xaa2-Xaa3-Xaa4-Xaa5-Xaa6-Xaa7-Z;
Xaa1 is not present or is selected from the group consisting of amino acid residues Asn, Thr, Phe, 3-(2-thienyl) alanine (Thi), 4-chlorophenylalanine (Cpa), α-naphthylalanine (α-Nal), β-naphthylalanine (β-Nal), 1,2,3,4-tetrahydronorharman-3-carboxylic acid (Tpi), Tyr, 3-iodo-tyrosine (o-l-Tyr), Trp and pentafluorophenylalanine (5-F-Phe) (all as L- or D-isomers);
Xaa2 is GIn, Asn or His;
Xaa3 is Trp or 1, 2, 3, 4-tetrahydronorharman-3-carboxylic acid (Tpi);
Xaa4 is Ala, Ser or Val;
Xaa5 is Val, Ser or Thr;
Xaa6 is Gly, sarcosine (Sar), D-Ala, or β-Ala;
Xaa7 is His or (3-methyl)histidine (3-Me)His;
Z is selected from —NHOH, —NHNH2, —NH-alkyl, —N(alkyl)2, and —O-alkyl
or Z is 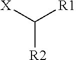
wherein X is NH (amide) or O (ester) and R1 and R2 are the same or different and selected from a proton, an optionally substituted alkyl, an optionally substituted alkyl ether, an aryl, an aryl ether or an alkyl-, halogen, hydroxyl or hydroxyalkyl substituted aryl or heteroaryl group; and,
a surfactant comprising a compound having (i) a polyethylene glycol chain and (ii) a fatty acid ester.

16. The pharmaceutical composition according to claim 15 wherein P is DPhe-Gln-Trp-Ala-Val-Gly-His-NH—CH(CH₂—CH(CH₃)₂)₂.

17. The pharmaceutical composition according to claim 15, wherein the GRPR-antagonist is NeoBOMB1 of formula (I): 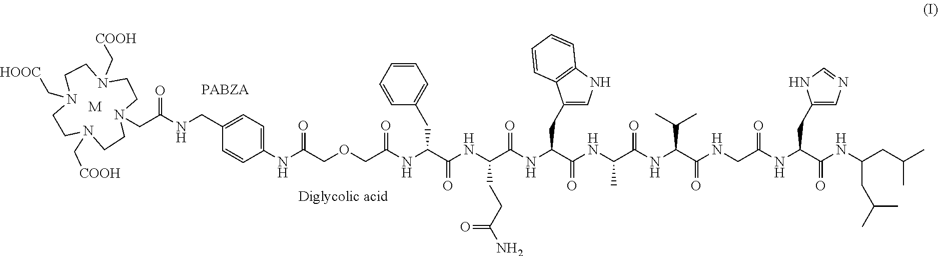
wherein M is a radiometal.

18. The pharmaceutical composition according to claim 17, wherein M is selected from ¹⁷⁷Lu, ⁶⁸Ga.

19. The pharmaceutical composition according to claim 15, wherein the surfactant comprises a compound of formula (III): 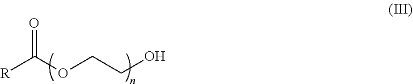
wherein n is comprised between 3 and 1000, and
R is the fatty acid chain.

20. The pharmaceutical composition according to claim 19, wherein n is comprised between 5 and 500.

21. The pharmaceutical composition according to claim 19, wherein n is comprised between 10 and 50.

22. The pharmaceutical composition according to claim 19, wherein R is an optionally substituted aliphatic chain 0.

23. The pharmaceutical composition according to claim 15, wherein the surfactant comprises polyethylene glycol 15-hydroxystearate and free ethylene glycol.

26. The pharmaceutical composition according to claim 15, wherein the radiolabeled GRPR-antagonist is present in a concentration providing a volumetric radioactivity of at least 100 MBq/mL.

27. The pharmaceutical composition according to claim 15, wherein the radiolabeled GRPR-antagonist is present in a concentration providing a volumetric radioactivity of between 250 MBq/mL and 500 MBq/mL.

28. The pharmaceutical composition according to claim 15, wherein the surfactant is present in a concentration of at least 5 μg/mL, optionally at least 25 μg/mL, and between 50 μg/mL and 1000 μg/mL.

29. The pharmaceutical composition according to claim 15, wherein the surfactant is present in a concentration of at least 25 μg/mL.

30. The pharmaceutical composition according to claim 15, wherein the surfactant is present in a concentration of between 50 μg/mL and 1000 μg/mL.

31. The pharmaceutical composition according to claim 15, wherein the radiolabeled GRPR-antagonist is labeled with ¹⁷⁷Lu, ⁶⁸Ga or ¹¹¹In.

32. The pharmaceutical composition according to claim 15, wherein the pharmaceutical composition is an aqueous solution.

33. The pharmaceutical composition according to claim 15, wherein the pharmaceutical composition is a solution for infusion.

34. A method for treating or preventing cancer in a subject in need thereof, the method comprising administering to said subject a therapeutically efficient amount of the pharmaceutical composition according to claim 15.

35. The method according to claim 34, wherein the therapeutically efficient amount comprises 2000 to 10000 MBq.

36. The method according to claim 34, wherein the therapeutically efficient amount of is administered to said subject 2 to 8 times per treatment.

37. A method for in vivo imaging of GRPR positive tumors in a subject in need thereof, the method comprising administering to said subject an effective amount of the pharmaceutical composition according to claim 15 and detecting the signal derived from the decay of the radioisotope present in said compound, thereby detecting GRPR positive tumors.
